(19) United States
(12) Patent Application Publication
(10) Pub. No.: US 2005/0209518 A1
(43) Pub. Date: Sep. 22, 2005

(54) Self-calibrating body analyte monitoring system

(75) Inventors: Burton H. Sage JR., Hot Springs, AR (US); David Gillett, Rancho Bemardo, CA (US); Brian Brandell, Chicago, IL (US)

Correspondence Address: Burton Sage, Jr.;c/o Therafuse, Inc., 2453 Impala Drive, Carlsbad, CA, 92008, US

(73) Assignee: Therafuse, Inc.

(21) Appl. No.: 11/083,362

(22) Filed: Mar. 16, 2005

Related U.S. Application Data
(60) Provisional application No. 60/553,564, filed on Mar. 17, 2004.

(51) Int. Cl. A61B 5/00; B65D 81/00

(52) U.S. Cl. 600/366

(57) ABSTRACT

A self-calibrating monitoring system based on microdialysis for measurement of a body analyte is disclosed. In one embodiment, perfusate containing a known concentration of body analyte is mixed with an enzyme solution after passing through a microdialysis needle and instead of passing through the microdialysis needle to measure the body analyte and to calibrate the analysis chamber that measures the body analyte.

This application claims priority to and subject matter disclosed in provisional application No. 60/553,564, filed on Mar. 17, 2004; the content of this application being incorporated by reference herein in its entirety. This application also claims subject matter disclosed in issued U.S. Pat. No. 6,582,393, issued Jun. 24, 2003, the contents of which are also incorporated by reference herein in their entirety.

### BACKGROUND OF THE INVENTION

A. Field of the Invention

The present invention relates in general to medical devices. Specifically, the invention relates to devices and methods for measuring the concentration of therapeutically useful compounds in body fluids.

B. Related Art

Microdialysis systems intended to measure the concentration of a body analyte, including systems to measure glucose, are known. In 1987 Lonnroth, et al published “A microdialysis method allowing characterization of intercellular water space in humans” in the American Journal of Physiology 253:E228-E231. Further, in 1995, Stemberg, et al published “Subcutaneous glucose in humans: real time estimation and continuous monitoring” in Diabetes Care 18:1266-1269.

The purpose of these efforts and devices, and the efforts and devices of many others, was to improve the methods of measuring glucose in blood and other body fluids, and thereby improve the quality of therapy for diabetes. In spite of these efforts, while significant progress has been made, there is yet no technological basis for a product based on microdialysis.

Many products are currently marketed to measure blood glucose. One class of these products, known as glucose strips and meters, require a blood sample, usually from a fingertip. They provide a satisfactory result when they are used, but they only provide a single result for each use. In diabetes, the glucose concentration in the body can change so quickly and so much that a single measurement, while being meaningful at the time it is taken, has little value a short time later. In general, the more frequently the glucose concentration is measured, the better diabetes can be managed. From a practical point of view, though, a new and accurate glucose measurement every three to five minutes is adequate to effectively manage even the most brittle cases of diabetes.

This need for more frequent glucose measurements has led to a class of glucose measuring systems (known as “needle” sensors) that monitor glucose continuously. For over two decades, devices of this class, that measure glucose in a blood vessel or in interstitial fluid just below the surface of the skin, have been under development. Recently, such a device for use in interstitial fluid, developed by the MiniMed Corporation, was approved for sale. It can be used for up to three days.

This product, and other “needle sensors” currently under development, must be calibrated by a blood glucose measurement, usually obtained from fingerstick blood using a “strip and meter” device. Current conventional wisdom holds that this need for calibration is due to a decrease in the sensitivity of the sensor over time during use. These sensors must be calibrated when the product is first placed in the skin and, in the case of the approved product, as frequently as every eight hours until it is removed. While this system does provide superior glucose information, it is much more inconvenient for the user, who must both insert the needle and provide calibration as needed from fingerstick glucose measurements.

To avoid the decrease in sensitivity with time exhibited by the “needle sensors”, microdialysis systems for glucose were developed. These systems moved the actual glucose detector from the tip of the needle sensor, which is inside the body, to a place outside the body. This change of location resulted in a much more stable glucose sensitivity. However, a microdialysis system is more complicated than a needle sensor, and early versions required perfusion of large volumes of fluid through the microdialysis needle, making the device too big for routine personal use. The volumes of fluids required for a day of use, for example, in the microdialysis system described by Pfeiffer in U.S. Pat. No. 5,640,954, were measured in hundreds of milliliters to liters per day. These early systems also separated the microdialysis needle from the assay location to such an extent that the time required for fluid exiting the microdialysis needle to reach the assay compartment was long, introducing a device related time lag. The time lag of these early systems could reach 30 minutes, a value too high to provide the best diabetes therapy.

Korf, in U.S. Pat. No. 6,013,029 describes an improved microdialysis system that uses much less fluid and also, in principle, reduces the time lag. In the preferred flow rate range specified by Korf, less than 20 microliters per hour, the amount of fluid required for a day's use is less than 480 microliters, a volume that can be very comfortably worn.

As advanced as Korf's system is, though, it still suffers from at least three problems. First, the flow through the system is continuous. Constant continuous flow of fluid, especially at the very slow flow rates described by Korf, is hard to establish and maintain. For example, the very low flow rates imply that the flow is driven by very low pressure differentials and driving forces. Thus even modest changes in atmospheric pressure, from weather systems or even from changes in altitude from, for example, traveling from Los Angeles to Denver, can result in significant flow rate changes. Also, for each of the fluid driving means described by Korf, as time passes, the flow rate will decrease. This happens as the fluid absorbing material is consumed, or due to backpressure developed in the capillary or behind the osmotic membrane, or through filling of the pressure differential reservoir. Korf makes no provision to compensate for this flow rate change, which can change the yield (see below) of his microdialysis device.

Second, a constant perfusate flow rate requires the body analyte to be measured by a sensor that measures the analyte by the rate at which a reaction occurs which in turn depends on the concentration of the analyte to be measured in the perfusate. Korf makes reference to an amperometric sensor that is sensitive to the concentration of hydrogen peroxide (or oxygen) present in the perfusate. These rate sensors are, by their nature, noisy and not totally accurate.

Third, Korf makes no provision for calibration of his system. At the very least, manufacturing variations will require that each system be calibrated before use. Also, no provision is made to accommodate variations in the degree of equilibrium achieved between the glucose concentration in the perfusate and the glucose concentration in the interstitial fluid. This degree of equilibrium is commonly referred to as yield. Yield varies directly with flow rate, implying the need for recalibration over time as the driving force is reduced.

Thus, while the system disclosed by Korf provides significant improvements over other older and larger microdialysis systems by dramatically reducing the volume of fluids, there is still room for improvement, especially in the area of calibration.

Sage, in patent publication 20030143746, describes a microdialysis system that includes a perfusate reservoir, a reagent solution reservoir for reacting with the selected body analyte, and an additional reservoir for a calibration solution. In this system, an analysis chamber is provided to alternate measurement of dialysate mixed with the reagent solution and calibration solution mixed with reagent solution. This system, however, has the disadvantage of the additional reservoir, which adds complexity to the system.

To resolve the additional complexity of a reservoir with a calibration solution, a known concentration of the selected body analyte may be added to the perfusate thereby eliminating the additional reservoir and fluid path. Adding the selected body analyte to the perfusate is well known in the art. In a technique known as “zero net flux”, the concentration of the selected body analyte in the perfusate is varied during use until the measured concentration in the dialysate is unchanged after microdialysis. In this condition, it is concluded that the tissue concentration of the body analyte is equal to the perfusate concentration of the body analyte since there was no change during microdialysis, that is, there was “zero net flux” of the body analyte into or out of the perfusate. Examples of the “zero net flux” method are provided by A. Le Quellec, et al in Microdialysis probes calibration: gradient and tissue dependent changes in no net flux and reverse dialysis methods, J Pharmacol Toxicol Methods 1995 Feb: 33(1): 11-16 or L. J. Petersen, et al in Microdialysis of the interstitial water space in human skin in vivo: quantitative measurement of cutaneous glucose concentrations, J Invest Dermatol 1992 Sept; 99(3): 357-60. These publications are incorporated herein in their entirety by reference.

However, the “zero net flux” method is difficult to implement in a commercial product since the concentration of the selected body analyte must be varied over time until equilibrium with tissue concentration is reached. This becomes a more complicated process and requires significant amounts of time per measurement—contrary to the desire for a continuous monitoring system. Pfeiffer and Hoss, in U.S. Pat. No. 6,091,976, describe a system with a constant concentration of the selected body analyte in the perfusate in order to calibrate the system. They further provide for non-continuous flow of the perfusate. During a first portion of the time, the system is operated at a low flow rate. During this low flow rate period, the yield of the selected body analyte is increased and the concentration of the selected body analyte in the dialysate is close to the tissue concentration. During a second portion of time, the system is operated at a high flow rate such that the concentration of the selected body analyte, after passing through the microdialysis needle, is essentially unchanged, thus providing a system calibration when the perfusate is analyzed during this second high flow rate period of operation. However, this method places high demand on the accuracy of the assay, since the concentration of the analyte in the tissue during the low flow rate portion of operation now must be calculated from the difference between the known concentration of glucose added to the perfusate and the concentration of glucose measured in the perfusate after microdialysis. When the assay is an enzyme catalyzed reaction, which is known to be subject to drift and temperature variations, the accuracy problem can be especially acute.

Further, the glucose containing perfusate that passes through the microdialysis needle during the high flow rate portion of operation will lose glucose to or gain glucose from the tissue, depending on the tissue concentration, thereby altering the concentration of the glucose in the perfusate somewhat. Hence, the accuracy of the “calibration” glucose concentration is questionable as well.

As can be seen from the issues and problems arising from prior art methods, there still remains a need for accurate, reliable, and convenient methods and systems to provide frequent measurement of body analytes.

### SUMMARY OF THE INVENTION

It is an object of this invention to provide a body analyte monitoring system with a self-calibration means so that the system may be used without the user obtaining and entering a calibration measurement at any time during its use. Accordingly, in one embodiment, a perfusate containing a known concentration of the body analyte is provided. The system also provides for two paths for perfusate to flow to a single measurement path—a first path from a perfusate reservoir through a microdialysis needle and a second path from the reservoir that bypasses the microdialysis needle. During a first segment of time, the body analyte laden perfusate proceeds down the first path to the microdialysis needle and flows through the microdialysis needle at such a rate that diffusion of the body analyte into the needle, in the case where the tissue concentration of the body analyte is higher than the concentration of the body analyte in the perfusate, or out of the needle, in the case where the tissue concentration of the body analyte is lower than the concentration in the perfusate, is in essential equilibrium, and the concentration of the body analyte in the dialysate (perfusate that has exited the microdialysis needle) is essentially equal to the concentration of the body analyte in the tissue. During this first segment of time, perfusate does not flow along the second path. After exiting the microdialysis needle, this perfusate proceeds to the measurement path where the concentration of the body analyte is measured.

During a second segment of time, the perfusate proceeds along the second path to the measurement path; this second path bypassing the microdialysis needle. During this second segment of time, perfusate does not flow along the first path through the microdialysis needle.

During the first time segment, flow of perfusate through the microdialysis needle proceeds at such a rate that when the perfusate emerges from the microdialysis needle as dialysate, the concentration of the body analyte in the dialysate is essentially equal to the concentration of the body analyte in the tissue. During the second time segment, flow of the perfusate in the microdialysis needle has stopped. However, diffusion of the body analyte into or out of the lumen of the microdialysis needle does not stop. Thus, the concentration of dialysate at the exit of the microdialysis needle is also essentially equal to the tissue concentration during the second time segment. In other words, under the stated flow condition, the concentration of the body analyte at the exit of the microdialysis needle is always essentially equal to the tissue concentration of the body analyte. Thus, flow of the perfusate along the first path may be stopped or started at will, knowing that at any time, dialysate from the microdialysis needle may proceed to the measurement path with a concentration essentially equal to the tissue concentration of the body analyte. At any time, then, the perfusate from the perfusate reservoir may be diverted along the second path to the measurement path and a measurement may be made of the known concentration of the body analyte. When this measurement is complete, flow may be restarted along the first path through the microdialysis needle and on to the measurement path such that a measurement of the concentration of the body analyte in the dialysate may be made.

In this manner of time-sharing the measurement path, the measurement path is calibrated by measuring the body analyte concentration in the perfusate that does not go through the microdialysis needle. This calibrates the measurement channel, thereby providing for accurate measurement of the body analyte concentration in the dialysate.

The measurement path may include a region where the dialysate undergoes exposure to an electric potential sufficiently high to oxidize or reduce any body substances which may interfere with measurement of the body analyte. In this case, the electric potential would be such that the body analyte would not be oxidized or reduced. If the electric potential is one where oxidation occurs, and the measurement method for the body analyte is one that requires oxygen to participate in the measurement, the potential may also be selected to be sufficiently high to electrolyze water, thereby creating oxygen which will then dissolve into the dialysate or perfusate.

The measurement path may include a region where the perfusate interacts with an immobilized enzyme. In this region, the interaction of the body analyte with the enzyme produces a product which may be analyzed to produce a signal proportional to the concentration of the body analyte in both the perfusate and dialysate. Alternatively, an enzyme solution from an enzyme reservoir may be mixed with the perfusate or dialysate along the measurement path. The enzyme will react with the body analyte producing a product which may be analyzed to produce a signal proportional to the concentration of the body analyte in the perfusate.

It is a further object of the invention to provide a body analyte monitoring system that minimizes the lag time, that is, the time required to obtain the sample and perform the assay of the concentration of a body analyte. In an embodiment of the invention, the microdialysis needle and the measurement path are all placed on a single substrate, thereby avoiding interconnects and additional plumbing that can increase the flow path length and hence the time required for the perfusate to travel from the exit of the microdialysis needle to the location where the measurement is made.

It is a further object of the invention to package the microdialysis system in a volume sufficiently small that it may be comfortably worn on the body, adhered to the body either by means of a strap or a skin adhesive.

### BRIEF DESCRIPTION OF THE DRAWINGS

FIG. 1 is a schematic of an embodiment of the body analyte monitoring system wherein the perfusate interacts with an immobilized enzyme.

FIG. 2 depicts a pressurized reservoir assembly of the embodiment of the invention shown in FIG. 1.

FIG. 3 is a schematic of an integrated microdialysis needle and measurement path including an immobilized enzyme of an embodiment of the invention.

FIG. 4 depicts a cross-section of a microdialysis needle of an embodiment of the invention.

FIG. 5 is a schematic of a fluidic controller of the embodiment of the invention shown in FIG. 1.

FIG. 6 is a schematic of a second embodiment of the body analyte monitoring system which includes an oxidation chamber.

FIG. 7 is a schematic of an integrated microdialysis needle and measurement path of the embodiment shown in FIG. 6.

FIG. 8 is a schematic of a third embodiment of the invention which includes a reservoir for a solution of an enzyme for reacting with the body analyte.

FIG. 9 is a schematic of an integrated microneedle and measurement path of the embodiment shown in FIG. 8.

FIG. 10 depicts a pressurized reservoir system for the embodiment of the invention shown in FIG. 8.

FIG. 11 is a schematic of a fluidic controller of the embodiment of the invention shown in FIG. 8.

FIG. 12 is an embodiment of the invention including a reservoir for an enzyme for reacting with the body analyte and an oxidation chamber.

FIG. 13 is a schematic of a fourth embodiment of the invention which includes both an oxidation chamber and a reservoir for an enzyme solution.

### DETAILED DESCRIPTION OF THE INVENTION

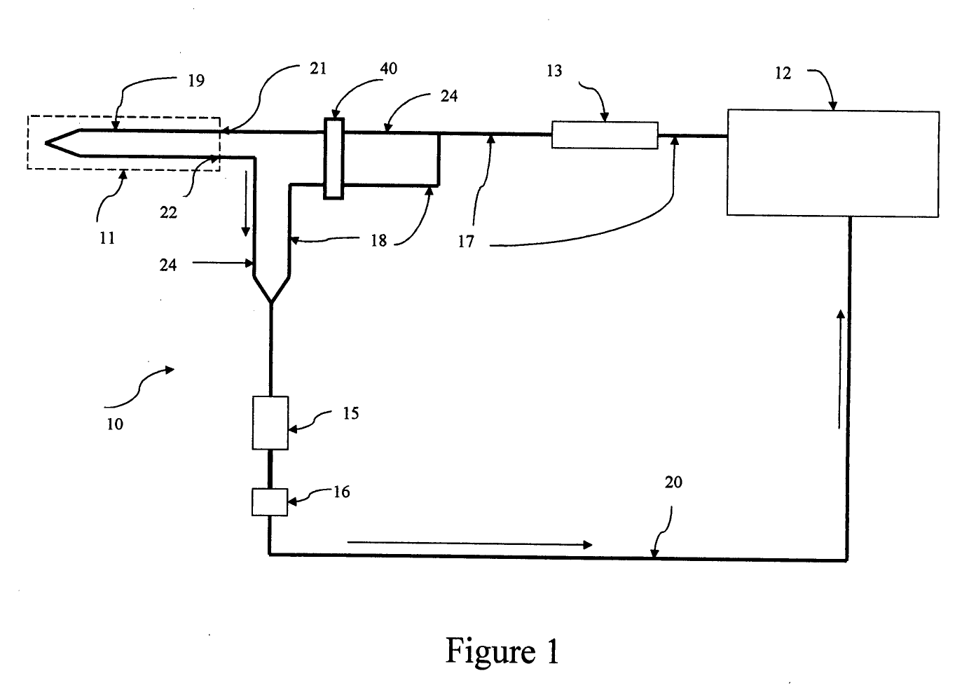

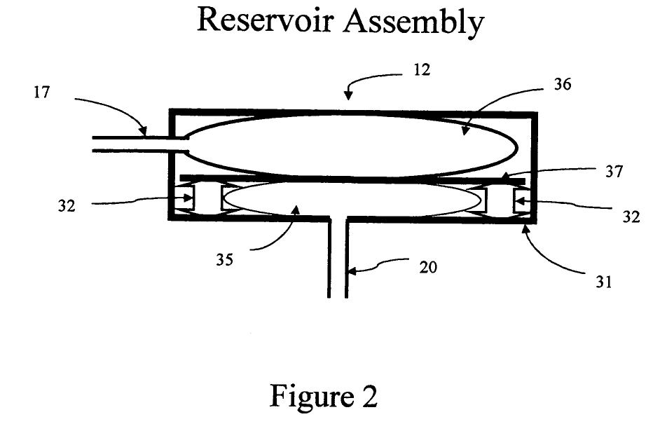

FIG. 1 shows a schematic of an embodiment of the invention. Microdialysis system 10 comprises perfusate supply system 12, flow restrictor 13, and flow paths 17 and 24 through valving system 40 which introduce the perfusate into the microdialysis needle 11 at inlet 21. Dialysate exits at outlet 22 and flows to measurement path 15 and 16. Flow paths 17 and 18 also pass the perfusate to measurement path 15 and 16 through the same valving system 40. The perfusate in supply system 12 (an example of a supply system is shown in FIG. 2) contains a known concentration of body analyte and may be an isotonic solution composed of saline and phosphate buffer, but may also contain other or different compounds to make the fluid biocompatible.

The perfusate contained in perfusate supply system 12 contains a known concentration of a body analyte. The body analyte may be glucose, or lactic acid, or any other chemical compound the tissue concentration of which may be desired. If the body analyte is glucose, the concentration of glucose in the perfusate may be in the range of 0.5 millimolar (9 milligrams per deciliter) to 50 millimolar (900 milligrams per deciliter) but is usually in the range of 2 millimolar (36 milligrams per deciliter) to 20 millimolar (360 milligrams per deciliter). A highly useful concentration of the body analyte in the perfusate when the body analyte is glucose is in the range of 3 millimolar (54 milligrams per deciliter) to 5 millimolar (90 milligrams per deciliter) because the accuracy of a glucose monitor should be highest at glucose concentrations wherein a state of hypoglycemic may be present or eminent.

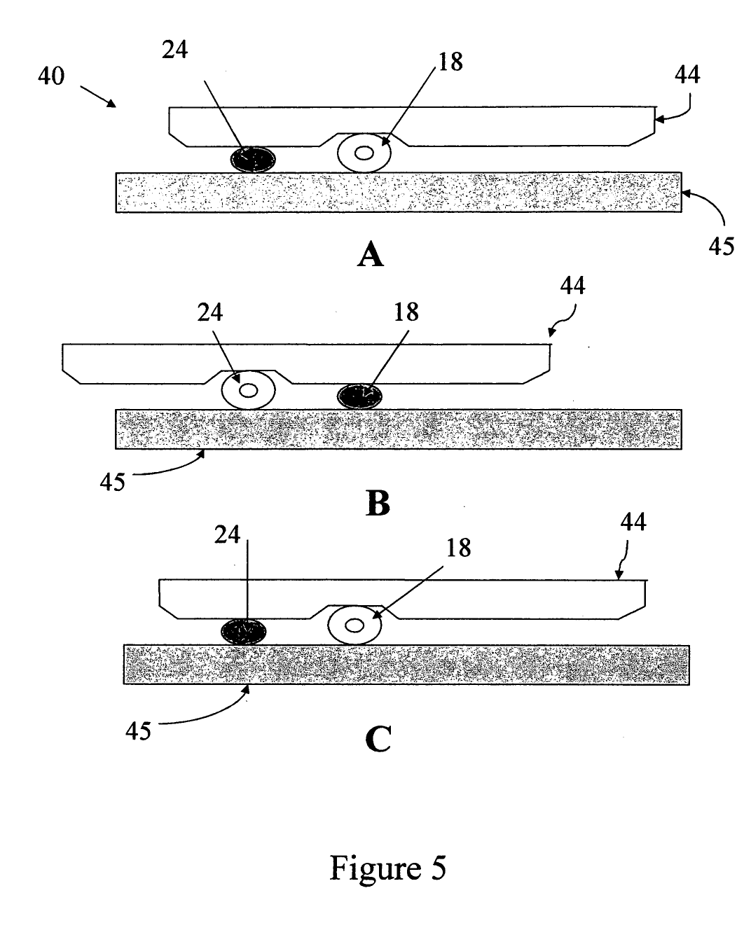

The perfusate exits supply system 12 via fluidic supply line 17 and passes through flow restrictor 13. Flow restrictor 13 may be a length of microbore tubing with an inside diameter selected to provide the desired flow rate. This inside diameter may be selected by the use of the Poiseuille equation or other means as is known in the art. Flow restrictor 13 may also be an orifice of a selected diameter to produce the desired flow rate as is also known in the art. After passing through flow restrictor 13, the perfusate continues to flow in fluidic flow line 17 to a “T” where the perfusate may flow along one of two paths—fluidic path 24 or fluidic path 18. The actual path along which the perfusate flows at any one time is selected by valving system 40 (one embodiment of valving system 40 is shown in greater detail in FIG. 5). When fluidic path 24 is selected by valving system 40, the perfusate flows into microdialysis needle 11 at inlet 21. The perfusate flows along lumen 19 of microdialysis needle 11 until it exits at outlet 22. One configuration of lumen 19 is shown in FIG. 4. In FIG. 4, lumen 19 is relatively wide and relatively shallow. A relatively shallow lumen is useful so that the time for the body analyte concentration in the tissue to equilibrate with the concentration of the body analyte at the bottom of the lumen should be short so that the transit time of the perfusate through microdialysis needle 11 is also short to minimize system lag (the time difference between the sample leaving the body and the time that the measurement of the body analyte concentration is complete). The time required for the body analyte to diffuse from the bottom of membrane 23 (FIG. 4) to the other side of lumen 19 (the shallow dimension of lumen 19) may be calculated using the following equation for the characteristic diffusion time t:
τ=χ²/D
where
τ=Diffusion characteristic time in seconds
χ=Depth of the lumen in centimeters
D=body analyte diffusion constant in cm²/Sec

For the concentration of the body analyte at the bottom of the channel of lumen 19 to be essentially equal to the concentration of the body analyte just below membrane 23, a time period of at least three characteristic times is needed. For glucose, the diffusion constant in solutions with a viscosity near that of water is 6.7×10⁻⁶ cm²/Sec. For a channel depth of one millimeter, the characteristic time τ can be calculated as nearly 1500 seconds. This would be way too long. For a depth of 0.1 millimeters, the characteristic time τ would be 15 seconds, which is much more reasonable. Therefore, useful depths of the lumen 19 are about 100 microns or smaller. A more highly useful depth would be 50 microns or smaller. When the channel is of sufficiently small size and the flow rate, in terms of speed along the channel is sufficiently slow, the overall volumetric flow rate of fluids is less than 1 nanoliter per second. The volume of fluid required to operate the system is then less than 100 microliters per day. This volume is sufficiently small that the entire system, including stored waste reagents, may be worn on the body, adhered thereto by a strap or skin adhesive.

Thus, for a given depth of lumen 19, a characteristic diffusion time may be calculated. In operation, then, perfusate enters the microdialysis needle with a body analyte concentration equal to the concentration in the perfusate reservoir. As the perfusate entering the microdialysis needle moves down lumen 19 towards the exit of the microdialysis needle, diffusion of the body analyte across membrane 23 (FIG. 4) occurs. It is useful to design the body analyte monitoring system such that the time that it takes an element of perfusate entering the microdialysis needle lumen 19 to travel completely along the lumen and exit the lumen, herein defined as the dwell time of the microdialysis needle, should be equal to or greater than three times the characteristic diffusion time. After passing through microdialysis lumen 19 and exiting microneedle assembly 11 at outlet 22, the perfusate proceeds along fluidic path 24 to immobilized enzyme chamber 15 and analysis chamber 16. These two chambers constitute a measurement path wherein a determination of the concentration of the body analyte is made. As an example, the body analyte may be glucose and the enzyme in chamber 15 is glucose oxidase, which reacts with glucose in chamber 15 to provide hydrogen peroxide which is easily measured by electrochemistry in chamber 16. But it should be understood that the body analyte may be any compound naturally found in the body or added to the body, and the enzyme may be any appropriately selected material to react with the body analyte to provide a reaction product easily measured. When the body analyte is glucose and the enzyme in chamber 15 is glucose oxidase, the hydrogen peroxide generated by the reaction of glucose with glucose oxidase in chamber 15 may be electrochemically reacted in chamber 16 in one of two ways. In a first way, the hydrogen peroxide may be measured amperometrically such that a current indicative of the hydrogen peroxide is continuously provided to a potentiostat as is well known in the art. Alternatively, the flow along measurement path 15 and 16 may be stopped and the hydrogen peroxide may be measured coulometrically. When coulometry is to be used, an electrical potential is provided for a period of time sufficient to react virtually all of the hydrogen peroxide in chamber 15. Potentials used for the electrochemical measurement of hydrogen peroxide are typically in the range of 300 millivolts to 800 millivolts.

As is also shown in FIG. 1, body analyte laden perfusate may also pass from reservoir 12 to measurement path 15 and 16 along flow path 18. Valving system 40, an example of which is shown in detail in FIG. 4, is configured to provide the perfusate from reservoir 12 either to chamber 15 through microdialysis needle 11 along flow path 24 or directly to chamber 15 in the measurement path along flow path 18. When flow path 18 is selected by valve system 40, perfusate having the known concentration is measured in measurement path 15 and 16. In this way the measurement path 15 and 16 is calibrated such that the signal, obtained when the known concentration of body analyte is measured, provides a timely reference factor. A new reference factor is obtained each time perfusate is measured so that changes in enzyme activity or other factors may be compensated. When valve system 40 is operated to alternate the fluid flowing into measurement path as, for example, first the perfusate from reservoir 12 and second dialysate from the outlet of microdialysis needle 11, highly accurate measurements of the dialysate are obtained since a fresh conversion factor from the perfusate is available for each subsequent measurement of the dialysate.

After measurement in chamber 16, the used fluids, either reacted perfusate or reacted dialysate, pass out of the measurement path along fluid path 20 to a waste reservoir in supply system 12.

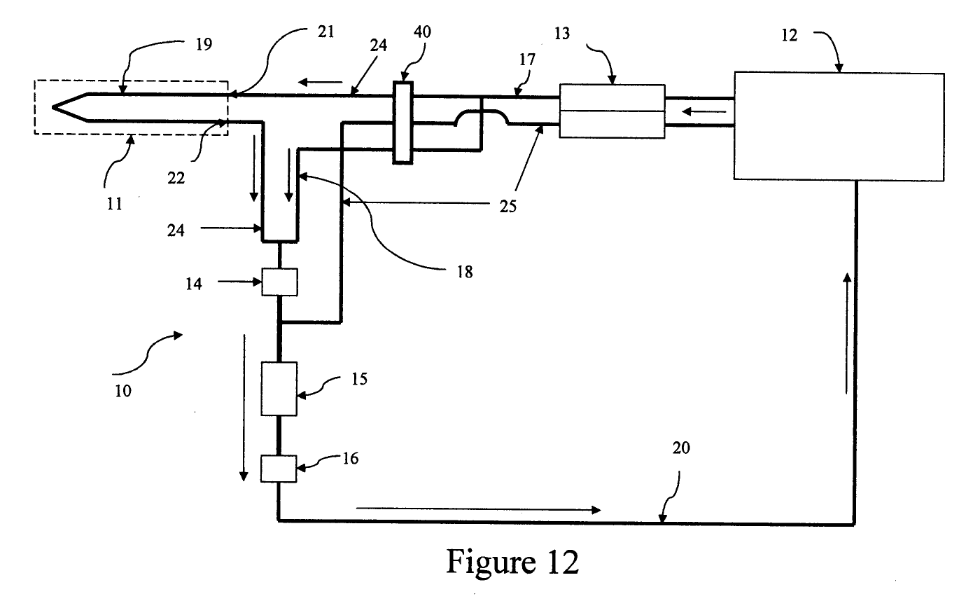

FIG. 2 shows an example of supply system 12 which may be used in the invention. The reservoir system is an assembly of five components—perfusate reservoir 36, waste reservoir 35, expandable spring 32, pressure plate 37 and housing 31. When reservoir 36 is full of perfusate, expandable spring 32 exerts pressure on reservoir 36 through pressure plate 37. The pressure exerted on reservoir 36 provides the driving force for causing the perfusate to flow along fluidic path 17 and thereby through microdialysis system 10. At the same time, expandable spring 32, physically attached to reservoir 35, provides a small pull on reservoir 35 and thereby analysis chamber 16 through fluidic path 20, thereby drawing the fluids that have passed through the measurement path 15 and 16 back to waste reservoir 35. Housing 31 provides the physical constraint enabling spring 32 to function as described. Reservoirs 35 and 36 may be plastic laminates with a biocompatible material such as polyethylene in contact with the fluid. Other layers in the laminate may be, as needed, a material such as PET for tensile strength and a light absorbing layer such as aluminum which may also function as a vapor barrier. Expandable spring 32 may be a wave spring, but may be a leaf spring of a spring of other configuration. The supply system assembly of FIG. 12 is but one example of how fluids may be moved through the microdialysis system. Fluid movement may also be caused by a variety of pumps including syringe pumps or peristaltic pumps or miniature butterfly pumps as is known in the art.

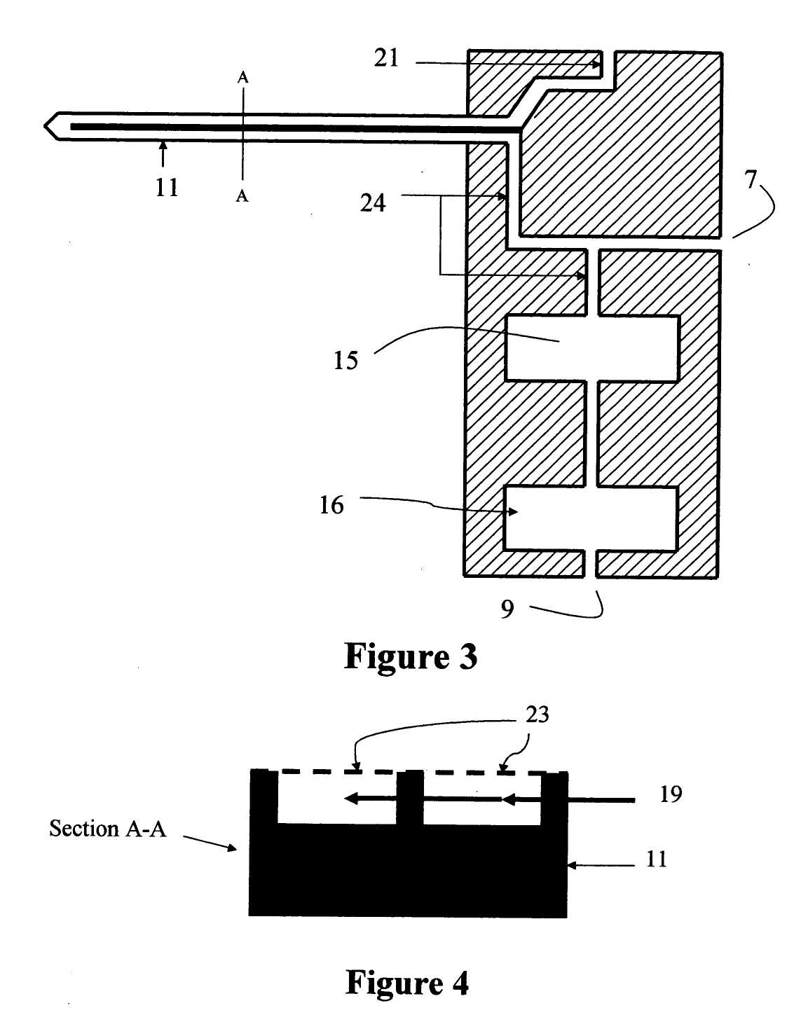

FIG. 3 shows an example of an integration of microdialysis needle 11 and measurement path 15 and 16 for microdialysis system 10. Microdialysis needle 11 may be an integral part of the unit as manufactured or microdialysis needle 11 and measurement chambers 15 & 16 may be manufactured separately and combined by assembly in a separate manufacturing step. The integrated unit has two inlets, inlet 21 and inlet 7, and one outlet 9. Inlet 21 allows perfusate from fluidic path 24 (FIG. 1) to flow into lumen 19 (FIG. 4) of microdialysis needle 11, exit lumen 19 by continuing flow path 24, and flow into measurement path 15 and 16. After analysis in measurement path 15 and 16, the spent fluid exits the integrated needle shown in FIG. 3 through exit 9.

The integrated assembly shown in FIG. 3 may be manufactured by a number of different techniques. Using MEMS (microelectromechanical systems) methods, the fluidic paths and chambers may be etched in a silicon substrate. Alternately, these fluidic paths and chambers may be formed on the surface of a substrate using photoresist or epoxy such as SU-8 or similar material. Using embossing techniques, these same fluidic paths and chambers may be formed on the surface of a polymer. In each of these cases, the fluidic paths and chambers may be covered with a second substrate on which the necessary electrodes are placed so that electrical contact may be made with the desired chambers. This second substrate may be of rigid materials such as glass or silicon or polycarbonate or other engineering polymers or may be of flexible materials such as polyimide or other materials used to manufacture flexible circuitry.

FIG. 4 shows a cross-section of microdialysis needle 11 and is an example of the geometry of microdialysis needle of the invention. Lumen 19 has been placed into a substrate by one of the methods described above. Microdialysis membrane 23 has been placed to cover lumen 19 so that when microdialysis needle 11 is in contact with a desired body fluid, the desired body analyte may migrate into lumen 19. Microdialysis membrane 23 may be made of cellulose acetate or polysulfone or other similar materials or may be a polycarbonate membrane with pores formed by the Track Etch process as is well known in the art. Microdialysis membrane 23 may cover only microdialysis needle 11 or may cover the entire integrated assembly including microdialysis needle 11, measurement path 15 and 16 and associated fluidic pathways.

FIG. 5 shows an example of valving system 40 in FIG. 1. Valving system 40 in FIG. 5 consists of two bars 44 and 45 between which are sandwiched flow tubes 24 and 18. Upper bar 44 may move back and forth horizontally between two positions as shown in FIGS. 5A and B. FIG. 5C is merely a repeat of FIG. 5A to show the return of valving system 40 to its original position after moving to the position shown in FIG. 5B. In the first position as shown in FIG. 5A, fluidic path 24 is pinched closed and fluid path 18 is open. Thus perfusate from reservoir assembly 12 will flow directly to measurement path 15 and 16, and a calibration measurement will be made by microdialysis system 10. In the second position shown in FIG. 5B, fluidic path 18 is pinched closed and fluidic path 24 is open. Thus perfusate from reservoir system 12 will flow along fluidic path 24 to the microdialysis needle where the body analyte in the perfusate will be exchanged with the body analyte in the tissue. After exiting the microdialysis needle at outlet 22, the dialysate will flow along measurement path 15 and 16 and a body analyte measurement will be made.

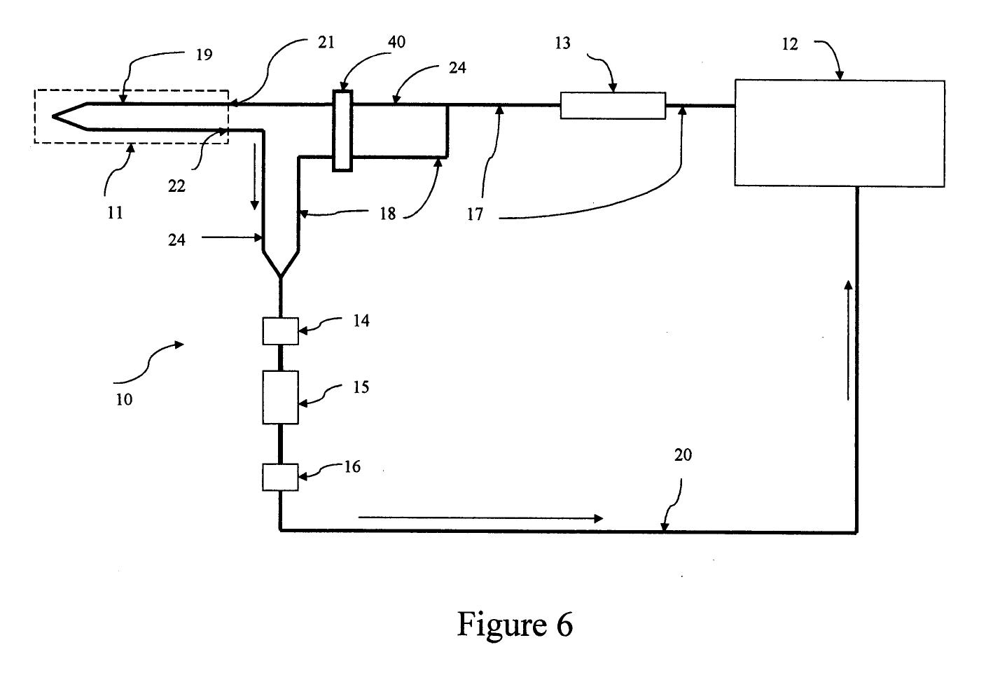

An alternative embodiment of the invention is shown in FIG. 6. By comparison to FIG. 1, it can be seen that the embodiment in FIG. 6 is identical to the embodiment in FIG. 1 except for the addition of oxidizer chamber 14 to measurement path 15 and 16 such that perfusate from flow path 18 or dialysate from flow path 24 both enter chamber 14 before flowing into chambers 15 and 16. For the purposes of this embodiment, the measurement path, previously defined as comprising chambers 15 and 16, will now comprise chambers 14, 15 and 16.

As in the embodiment shown in FIG. 1, perfusate from supply system 12 flows along flow path 17 to the “T” where the perfusate may either flow along flow path 24 or along flow path 18, depending on the state of valving system 40. When valving system 40 is set to permit perfusate to flow along flow path 24, the perfusate flows to microdialysis needle 19 and exits at outlet 22 as dialysate.

The dialysate proceeds along fluidic path 24 to oxidizer chamber 14, immobilized enzyme chamber 15 and analysis chamber 16. These three chambers comprise the measurement path wherein the concentration of the body analyte in the dialysate or perfusate is measured. In this measurement process, chamber 14 plays the role of reducing potential interferents and may introduce oxygen (depending on operation parameters) into the perfusate or dialysate if the reaction of the body analyte with the immobilized enzyme in chamber 15 requires oxygen and there is the potential for insufficient oxygen in the perfusate or dialysate. Chamber 15 contains an immobilized enzyme that reacts with the body analyte (and oxygen if needed) to create a molecule which is more readily analyzed. For example, if the body analyte is glucose and the enzyme is glucose oxidase, then hydrogen peroxide is the reaction product which is readily analyzed electrochemically as is well known in the art. Chamber 16 is the analysis chamber where the reaction product is analyzed. If the reaction product is electrochemically active, then chamber 15 is an electrochemical cell. If the reaction product is optically active, then chamber 15 is an optical absorption or fluorescence cell.

The above paragraphs describe the functioning of this second embodiment of microdialysis system 10 when the perfusate progresses from reservoir system 12 to measurement path 14, 15, and 16 along flow paths 17 and 24. In this mode, the dialysate exiting outlet 22 contains a concentration of the body analyte essentially equal to the tissue concentration of the body analyte. Alternatively, perfusate may progress from reservoir system 12 to measurement path 14, 15, and 16 along fluidic path 17 and 18 which bypasses microdialysis needle 11. Valving assembly 40 alternatively selects fluidic path 18 or fluidic path 24. Perfusate moving to measurement path 14, 15, and 16 along flow path 18 has bypassed the microdialysis needle and therefore contains the original concentration of the body analyte. Thus when perfusate from fluidic path 18 enters the measurement path, the measurement process provides an output for which the input is known. In this way, a measurement of the perfusate from fluidic path 18 constitutes a calibration for the measurement of the dialysate that progresses to the measurement path along path 24 as was discussed with regard to the embodiment shown in FIG. 1.

In a further embodiment of the invention, chamber 14 is both an oxidizer and oxygenator. Chamber 14 is supplied with an electrode in contact with the perfusate that has a potential of 1.22 volts or slightly greater. In Chamber 14 with an electrode at this potential, compounds which are oxidizable at the same potential as electrochemically active reaction products that would be reacted in electrochemical cell 16 are eliminated before the body analyte is reacted into an electrochemically active product in chamber 15. In the case that the body analyte is glucose, the electrochemically active product is hydrogen peroxide which is electrochemically active at potentials above about 350 millivolts. The hydrogen peroxide is created in chamber 15, after the perfusate has passed through chamber 14. Since glucose is not electrochemically active at about 1.22 volts or slightly greater, only those compounds in the perfusate which may interfere with the measurement of the body analyte are oxidized, removing these potential interferents. By placing the potential at or slightly higher than 1.22 volts, oxygen is also added to the perfusate through the well known electrolysis process. While there may or may not be sufficient oxygen in the perfusate to complete the reaction between the body analyte and the enzyme in chamber 15, creating and adding oxygen to the perfusate in chamber 14 insures that adequate oxygen is available.

Chamber 15 comprises the reaction chamber in this embodiment. The body analyte molecules in the perfusate moving along fluidic path 24 or fluidic path 18 pass through chamber 14 unperturbed. Upon entering reaction chamber 15, the body analytes reacts with the enzyme to form desired reaction products as discussed above. These reaction products move out of chamber 15 to analysis chamber 16. In an embodiment where chamber 16 is designed to perform an electrochemical analysis, chamber 16 comprises an electrode which is in contact with the fluid in chamber 16, which is either perfusate or dialysate. This electrode is set to a potential for reacting with the selected reaction product from chamber 15. In the case of glucose, the reaction product is hydrogen peroxide, which is oxidized to oxygen and water with the release of two electrons when the potential of the electrode is sufficient. Useful values for the potential of the electrode are well known in the art, and range from a lower value near 300 millivolts to over 700 millivolts. Alternately, chamber 16 may be an optical analysis chamber when the desired reaction product may be detected optically.

When chamber 16 is an electrochemical cell, the reaction product in chamber 16 may be measured in one of two ways. In a first way, fluid flow through the system is not stopped. Using the well known amperometric method, at a particular point in time, the electrode in chamber 16 is changed from zero volts to its operating voltage for a predetermined length of time. The current flowing with this voltage set at the selected value follows the well known Cottrell profile. This current profile is stored for both dialysate from the microdialysis needle flowing along fluid path 24 and for perfusate flowing along fluid path 18 for calibration. By analyzing the current profile, a value for the concentration of the body analyte can be calculated. Alternatively, using the Coulometric approach, the perfusate may be stopped and the electrode in chamber 16 set to its operating point for a length of time sufficient to react essentially all of the hydrogen peroxide in chamber 16. After reaction in analysis chamber 16, the perfusate passes along fluidic path 20 to a waste container in reservoir system 12.

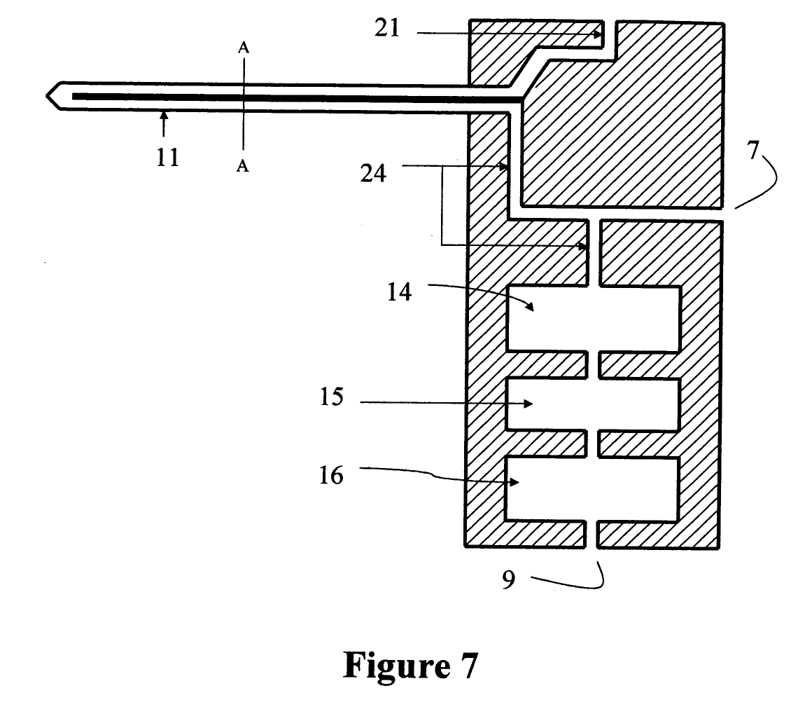

As was the case for the embodiment shown in FIG. 1, the microdialysis needle and measurement path may be integrated onto a single substrate. An integrated microdialysis needle and measurement path for the embodiment shown in FIG. 6 is shown in FIG. 7. Chamber 14 has been added such that perfusate from flow path 18 enters at inlet 7 or dialysate from outlet 22 of the microdialysis needle enter this chamber before proceeding to chambers 15 and 16. The integrate microdialysis needle and measurement path may be manufactured on the same substrate as for the embodiment shown in FIG. 1, or they may be manufactured separately and assembled onto the same substrate. Fluid that has passed through measurement path 14, 15 and 16 exits the measurement path at outlet 9 and proceeds to a waste reservoir. For the embodiment shown in FIG. 6, the supply reservoir system 12 and valve assembly 40 are as shown in FIGS. 2 and 5 respectively, and function as described with respect to these figures.

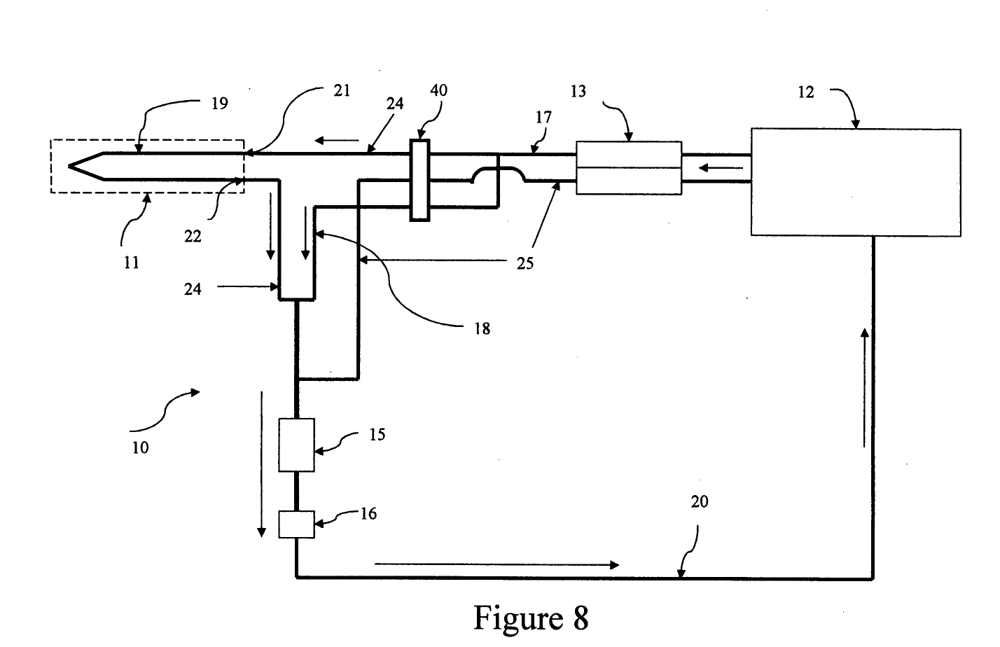

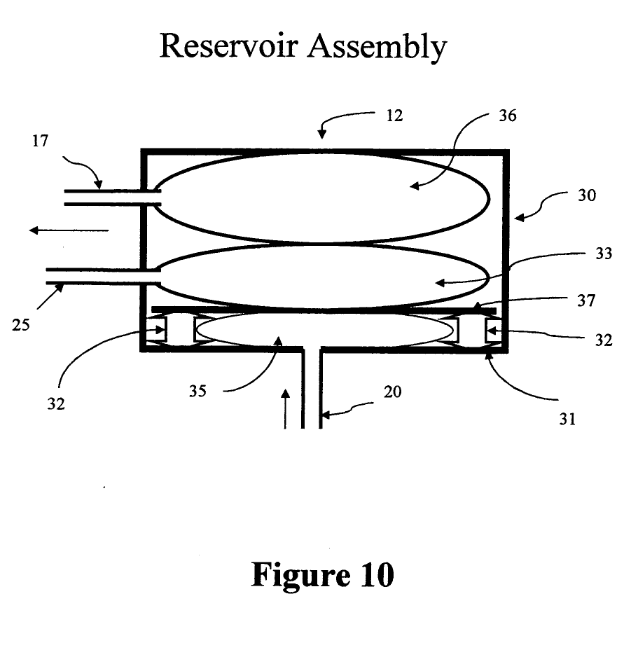

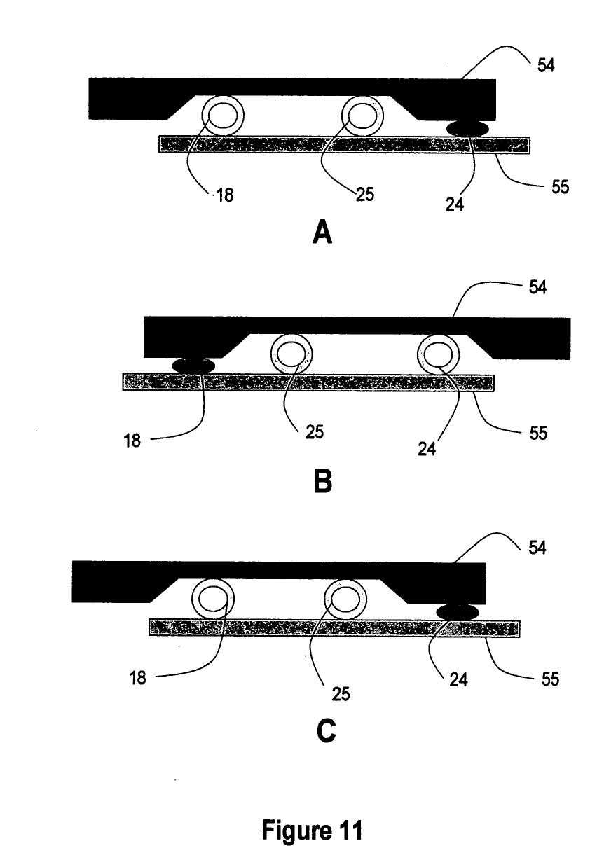

A further embodiment of the invention is shown in FIG. 8. Operation is also very similar to that described in the embodiment shown in FIG. 1. The difference in this case is the omission of the immobilized enzyme in chamber 15 and replacement of that function by a solution of enzyme for reacting with the body analyte. To accommodate this change, the following changes are made as shown in FIG. 8. Reservoir supply system 12 requires an additional reservoir for containing the enzyme solution. The changes to the reservoir for this embodiment are shown in FIG. 10. Flow resistor 13 requires an additional path. Instead of a single resistive path, there are now two. The additional path may be an extra lumen in a single component, or an additional single lumen resistor may be added. Fluidic path 25 has been added to conduct the enzyme solution to measurement path 15 and 16. Fluidic path 25 must further be accommodated in valving system 40 as described below. The final change relates to measurement path 15, and 16. Both the calibration perfusate from fluidic path 18 and the tissue dialysate from outlet 22 of the microdialysis needle eventually flow to measurement path 15 and 16 as before. In this case immobilized enzyme chamber 15 in FIG. 1 is replaced by enzyme mixing chamber 15 in FIG. 8. Valving assembly 40, similar to that shown in FIG. 5 except that it now functions with three tubes instead of two, alternately permits flow from fluidic paths 25 and 24 or fluidic paths 25 and 18 as shown in FIG. 11. When fluidic paths 25 and 24 are selected, the perfusate that has passed through microdialysis needle 11 enters measurement path 15 and 16. The dialysate exiting microdialysis needle 11 at exit 22 contains a concentration of body analyte essentially equivalent to that of the body tissue. This dialysate mixes with enzyme solution flowing in fluidic path 25 to provide the reaction product that is measured in analysis chamber 16. When fluidic paths 25 and 18 are selected, perfusate that has not passed through microdialysis needle 11 flows towards mixing chamber 15. Just before entering chamber 15, enzyme from flow path 15 is added to the perfusate. These reagents react to provide the reaction product that is measured in analysis chamber 16. As in FIG. 1, fluids flowing out of analysis chamber 16 are then collected in the waste reservoir of reservoir assembly 12 by flowing along fluidic path 20.

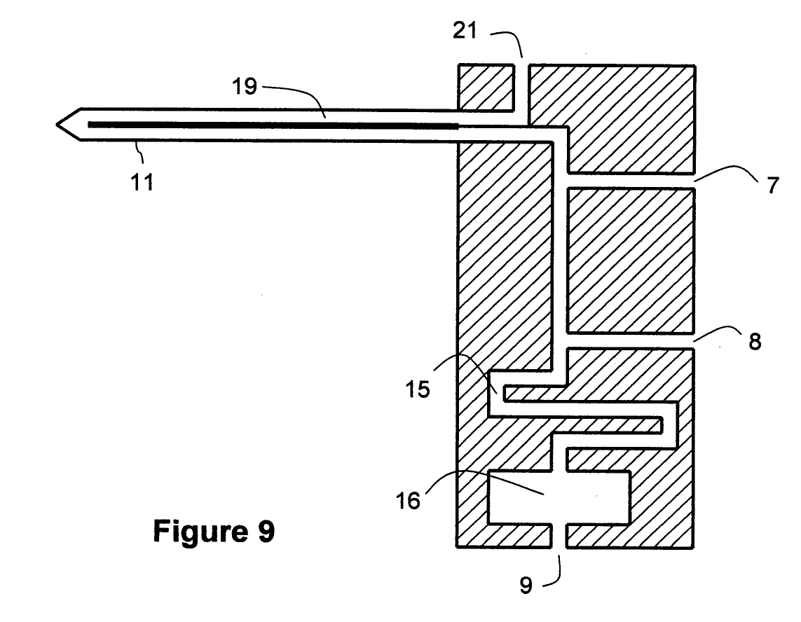

As was the case for the embodiments shown in FIG. 1 and 6, the embodiment shown in FIG. 8 can also be integrated so that the microdialysis needle and the measurement path are on a single substrate. This integration is shown in FIG. 9. The integration is very similar to that shown in FIG. 3, with the exception that chamber 15 no longer contains immobilized enzyme but is a mixing chamber for body analyte laden fluid entering measurement path 15 and 16 at inlet 21 or inlet 7. Mixing chamber 15 may be a tortuous path as shown in FIG. 9 or may be of another geometrical shape as is known in the art. When the body analyte is glucose and the enzyme is glucose oxidase, the dwell time for the interaction of the glucose oxidase and glucose is sufficiently long to permit essentially complete reaction of the glucose oxidase with glucose, but is not so long that mutarotation of the alpha form of glucose to the beta form begins to be a factor. Analysis for the reaction product occurs in chamber 16 as in the first embodiment.

The alternative embodiment shown in FIG. 8 requires a modified reservoir system 12. This modified reservoir system is shown in FIG. 10. As before, it contains the body analyte laden perfusate in reservoir 36, fluidic path 17 for carrying the perfusate to the microdialysis needle and measurement path, expanding spring 32, pressure plate 37, waste reservoir 35 with connecting fluidic path 20, and housing 31. The added component is enzyme solution reservoir 33 and connecting fluidic path 25 for carrying the enzyme solution to the measurement path. As in the first embodiment, expanding spring 32 puts mechanical pressure on reservoirs 33 and 35 causing the fluids to flow from the reservoirs. In addition, expanding spring 32 puts a small pull on waste reservoir 35 to help draw fluids from the reaction chamber 16 along fluidic path 20 into the waste reservoir.

The alternative embodiment shown in FIG. 8 also requires a modified valving system 40. This modified valving system is shown in FIG. 11. As in FIG. 5, valving system 40 comprises two horizontal bars, shown as 54 and 55 in FIG. 11. Upper bar 54 moves horizontally with respect to bar 55, and can alternately pinch off flow paths 24 and 18 as shown in FIGS. 11A and 11B. As mentioned above, valving system 40 regulates flow in three flow paths-flow paths 18, 24, and 25. During the period of time when a calibration measurement of perfusate is being made, valving system 40 allows flow along flow paths 18 and 25, as is shown in FIG. 11A. When a measurement of the body analyte is being made, valving system 40 allows flow along flow paths 24 and 25 as is shown in FIG. 11B. FIG. 11C merely shows the return of valving system 40 to the initial configuration shown in FIG. 11A to begin another cycle of perfusate measurement followed by body analyte measurement.

FIG. 12 shows yet another embodiment of the invention. As was described with regard to the embodiment shown in FIG. 6, oxidation chamber 14 has been added to eliminate potential interferents. This chamber functions in the same way as the oxidation chamber shown in FIG. 6. Flow path 25 enters measurement path after oxidation chamber 14 but before mixing chamber 15 to avoid reaction products generated by the reaction of the body analyte and the enzyme being oxidized in chamber 14. Valving system 40 and reservoir system 12 function in the same way as described regarding the embodiment shown in 8.

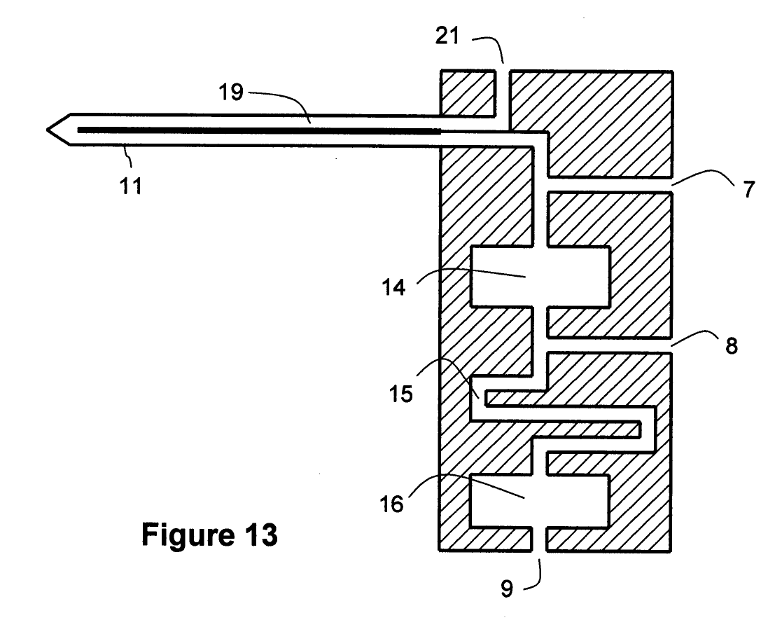

As was the case for the other embodiments described above, the embodiment shown in FIG. 12 can also be reduced in size and integrated onto a single substrate. This integration is shown in FIG. 13. This integrated assembly has three inlets—inlets 7 and 8, where perfusate from reservoir system 12 enters, and inlet 21, where enzyme solution enters. Perfusate from inlet 7 and dialysate from the outlet of microdialysis needle pass into oxidation chamber 14 where oxidizable interferents are removed, and, if necessary, oxygen is added. After exiting from oxidation chamber 14, these fluids receive enzyme solution from inlet 8, and are mixed in mixing chamber 15, where the reaction products are generated. The reaction products are measured in analysis chamber 16 as has been previously described. Spent fluids exit at outlet 9 and are captured in the waste reservoir of supply system 12 after flowing along flow path 20.

It is noted that the phrase “in liquid communication” is used herein. By “in liquid communication,” it is meant that components of the devices described herein modified by the phrase are connected to each other by liquid passages. The fact that a valve or other flow blocking/flow diverting component may lie between two components or points that are in fluid communication with each other, even when closed, does not take these components/points out of fluid communication with each other.

In view of the above, some embodiments of the Body Analyte Monitoring System Assembly as described above and/or according to other embodiments of the present invention may be used in combination with one or more of the embodiments of the drug delivery systems described in U.S. application Ser. No. 10/146,588 dated May 15, 2002 and/or U.S. application Ser. No. 10/600,296 dated Jun. 20, 2003, and/or copending application Ser. No. 10/059,390, filed Jan. 31, 2002, and/or U.S. application Ser. No. 09/867,003 filed May 29, 2001, now U.S. Pat. No. 6,582,393, issued Jun. 24, 2003, and/or U.S. application Ser. No. 10/662,871 dated Sep. 16, 2003, and/or copending application Ser. No. and/or copending application Ser. No. 10/786,562 filed on Feb. 26, 2004, and/or provisional application No. 60/553,564 filed on Mar. 17, 2004. Thus, some embodiments of the present invention include the combination of a body analyte monitoring system/self-calibrating body analyte monitoring system utilizing the Body Analyte Monitoring System Assembly as disclosed herein in combination with a drug delivery system, which may be, by way of example and not by way of limitation, in a single integrated system and/or in two or more quasi-separate systems in communication with each other which may, again by example, be worn or otherwise carried by a user. In such embodiments, a Body Analyte Monitoring System Assembly as described herein may be utilized in or with a body analyte monitoring system/self-calibrating body analyte monitoring system to monitor a body analyte and/or a drug delivery system to control the amount/rate/dosage, etc., of drug delivered to the user based on the results of monitoring by the body analyte monitoring system utilizing the Body Analyte Monitoring System Assembly. Thus, in some embodiments, a device/method may be manufactured/used where the two systems/assemblies work together to ensure/help ensure that a patient receives proper/adequate amounts of a beneficial drug.

While specific embodiments of the invention have been described in detail, it will be appreciated by those skilled in the art that various modifications and alternatives to those details could be developed in light of the teaching of the disclosure. Accordingly, the particular embodiment described in detail is meant to be illustrative and not limiting as to the scope of the invention, which is to be given the full breadth of the appended claims and any and all equivalents thereof

1. A device for measuring a concentration of a body analyte concentration comprising:
a) a reservoir containing a perfusate comprising a known concentration of the body analyte,
b) an interface including an inlet in liquid communication with the reservoir and an outlet, wherein the interface is adapted to allow exchange of the body analyte between the perfusate and the body fluid when there is perfusate in the interface and the interface is in contact with the body fluid,
c) a measurement path for measuring the body analyte concentration downstream from the outlet and the reservoir such that an entrance to the measurement path is in liquid communication with both the reservoir and the outlet of the interface, and
d) a valving system for controlling flow into the measurement path such that liquid flowing into the measurement path is either liquid from the reservoir or liquid from the outlet.

2. The device of claim 1 wherein the flow of the perfusate through the interface is such that the dwell time of the perfusate in the interface is greater than three times the characteristic diffusion time of the body analyte in the interface.

3. The device of claim 1 wherein the interface and the measurement path reside on a common support.

4. The device of claim 1 wherein the interface has a lumen along which the perfusate flows, the lumen being rectangular in section with a depth less than 100 microns.

5. The device of claim 1 wherein the measurement path comprises a first chamber and a second chamber downstream from the first chamber.

6. The device of claim 5 wherein the first chamber comprises an immobilized enzyme.

7. The device of claim 5 wherein the device is adapted so that a measurement related to the concentration of the body analyte is made at the second chamber.

8. The device of claim 1 wherein the measurement path comprises a first chamber, a second chamber downstream from the first chamber, and a third chamber downstream from the second chamber.

9. The device of claim 8 wherein the interface and the measurement path reside on a common support.

10. The device of claim 9 wherein the interface has a lumen along which the solution flows, the lumen being rectangular in section with a depth less than 100 microns.

11. The device of claim 8 wherein the first chamber comprises an oxidation chamber.

12. The device of claim 8 wherein the second chamber comprises an immobilized enzyme.

13. The device of claim 8 wherein the third chamber comprises a measurement chamber.

14. The device of claim 11 wherein the oxidation chamber both removes potential interferents and generates oxygen.

15. The device of claim 13 wherein the measurement chamber is a body analyte concentration measurement chamber

17. A device for measuring a concentration of a body analyte in a body fluid, comprising:
a) a first reservoir containing a perfusate comprising a known concentration of the body analyte,
b) a second reservoir containing an enzyme solution,
c) an interface including an inlet in liquid communication with the first reservoir and an outlet, wherein the interface is adapted to allow exchange of the body analyte between the perfusate and the body fluid when there is perfusate in the interface and the interface is in contact with the body fluid,
c) a measurement path for measuring the body analyte concentration downstream from the outlet and the reservoir such that an entrance of the measurement path is in liquid communication with the first reservoir, the second reservoir, and the outlet of the interface, and
d) a valving system for controlling flow into the measurement path such that the liquid flowing into the measurement path is either perfusate from the reservoir mixed with the enzyme solution or dialysate from the outlet mixed with the enzyme solution.

18. The device of claim 17 wherein the flow of the perfusate through the interface is such that the dwell time of the perfusate in the interface is greater than three times the characteristic diffusion time of the body analyte.

19. The device of claim 17 wherein the interface and the measurement path reside on a common support.

20. The device of claim 17 wherein the interface has a lumen along which the perfusate flows, the lumen being rectangular in section with a depth less than 100 microns.

21. The device of claim 17 wherein the measurement path comprises a first chamber and a second chamber downstream from the first chamber.

22. The device of claim 21 wherein the first chamber is adapted to mix the dialysate from the outlet and the solution from the second reservoir.

23. The device of claim 21 wherein the second chamber is a body analyte concentration measurement chamber.

24. A device for measuring a concentration of a body analyte in a body fluid, comprising:
a) a first reservoir for containing a perfusate comprising a known concentration of the body analyte,
b) a second reservoir for containing an enzyme solution,
c) an interface including an inlet in liquid communication with the first reservoir and an outlet, wherein the interface is adapted to allow exchange of the body analyte between the perfusate and the body fluid when there is perfusate in the interface and the interface is in contact with the body fluid,
c) a measurement path comprising a first chamber, a second chamber downstream from the first chamber, and a third chamber downstream from the second chamber such that the first chamber may receive perfusate from the first reservoir and the dialysate from the outlet and the second chamber may receive either perfusate or dialysate from the first chamber and enzyme solution from the second reservoir, and
d) a valving system for controlling liquid flow along the measurement path such that the liquid flowing into the second chamber is either (i) dialysate from the outlet that has passed through the first chamber and enzyme solution from the second reservoir, or (ii) perfusate from the first reservoir that has passed through the first chamber and enzyme solution from the second reservoir.

25. The device of claim 24 wherein the interface and the measurement path reside on a common support.

26. The device of claim 24 wherein the interface has a lumen along which the perfusate flows, the lumen being rectangular in section with a depth less than 100 microns.

27. The device of claim 24 wherein the first chamber is an oxidation chamber.

28. The device of claim 27 wherein the oxidation chamber both removes potential interferents and generates oxygen.

29. The device of claim 24 wherein mixing of perfusate or dialysate from the first chamber with enzyme solution from the second reservoir occurs in the second chamber.

30. The device of claim 24 wherein the device is adapted so that a measurement relating to the concentration of the body analyte is made at the third chamber.

31. A microdialysis based body analyte monitoring system comprising:
an electrochemical chamber to process dialysate operating at a potential to oxidize potential body analyte interferents and oxidize water to oxygen.

32. A method of calibrating a microdialysis based body analyte monitoring system comprising:
providing a perfusate solution with a known concentration of the body analyte and alternating an analysis of a concentration of the body analyte of (a) dialysate that has passed through an interface in contact with a body fluid such that diffusion of the body analyte into or out of the interface has reached essential equilibrium before exiting the interface, and (b) perfusate that has bypassed the interface such that it retains its original body analyte concentration.

33. The method of claim 32, further comprising:
modifying the results of the analysis of the body analyte of the dialysate that has passed through the interface in contact with the body fluid such that diffusion of the body analyte into or out of the interface has reached essential equilibrium before exiting the interface, wherein modification of the results includes scaling the results according to a comparison of (i) the results of the analysis of the perfusate that has bypassed the interface such that it retains its original body analyte concentration and (ii) the known concentration of the body analyte.

34. A method of providing oxygen to the measurement path of the microdialysis based body analyte monitoring system comprising the steps of a) providing an oxidation chamber in the measurement path, and b) operating the oxidation chamber at a potential sufficient to electrolyze water.

35. A method of operating a microdialysis based body analyte monitoring system including the step of electrochemically processing dialysate at a potential sufficiently high to oxidize potential body analyte interferents in the dialysate and to oxidize water to oxygen.

36. A device for measuring a concentration of a body analyte in a body fluid, comprising:
a) a reservoir containing a perfusate comprising a known concentration of the body analyte;
b) a lumen adapted to be inserted into human skin, the lumen including a liquid flow path, wherein the lumen is further adapted to permit the diffusion of the body analyte into the liquid flow path and out of the liquid flow path when the lumen lies inserted into human skin and in contact with body fluid;
c) a valving system, wherein the valving system is adapted to alternately direct perfusate from the reservoir (i) through the liquid flow path in the lumen and then through a measurement path and (ii) to bypass the lumen and flow through the measurement path; and
d) a microcontroller/microprocessor assembly adapted to control the valving system to alternately direct perfusate from the reservoir (i) through the liquid flow path in the lumen and then through the measurement path and (ii) to bypass the lumen and flow through the measurement path;
wherein the device is adapted to measure the body analyte concentration of the perfusate passing through the measurement path,
wherein the microcontroller/microprocessor assembly is further adapted to compare (1) a first value of a measurement of the body analyte concentration of perfusate in the measurement path that has been directed to bypass the lumen and flow through the measurement path to (2) the known concentration of the body analyte in the perfusate of the reservoir;
wherein the microcontroller/microprocessor assembly is further adapted to identify a conversion factor based on the comparison of (1) and (2); and
wherein the microcontroller/microprocessor assembly is further adapted to obtain a second value of a measurement of the body analyte concentration of perfusate in the measurement path that has been directed through the lumen and then into the measurement path and output a modified body analyte concentration value, the modified body analyte concentration value being the second value as modified by the conversion factor.

37. The device of claim 36, wherein the device is adapted to be worn on a human body part.

38. A device for measuring a concentration of a body analyte in a body fluid, comprising:
a lumen adapted to be inserted into human skin, the lumen including a liquid flow path, wherein the lumen is further adapted to permit the diffusion of the body analyte into the liquid flow path and out of the liquid flow path when the lumen lies inserted into human skin and is in contact with body fluid; and
a measurement assembly adapted to measure the concentration of body analyte in a fluid passing through a measurement path;
wherein the device is adapted to alternately (i) pass perfusate containing a known concentration of the body analyte through the lumen and through the measurement path and (ii) to bypass the lumen and direct the perfusate to through the measurement path;
wherein the device is further adapted to calibrate itself by comparing the measured concentration of the body analyte in the fluid passing through the measurement path to the known concentration of the body analyte and modify the measured value of the concentration of body analyte in the perfusate that has passed through the lumen and through the measurement path based on this comparison.

39. The device of claim 1 further comprising a drug delivery device such that the amount or rate of drug delivery by the drug delivery device is based on a measurement made by the device.

40. The device of claim 17 further comprising a drug delivery device such that the amount or rate of drug delivery by the drug delivery device is based on a measurement made by the device.

41. The device of claim 24 further comprising a drug delivery device such that the amount or rate of drug delivery by the drug delivery device is based on a measurement made by the device.
